(19) United States
(12) Patent Application Publication
(10) Pub. No.: US 2021/0158922 A1
(43) Pub. Date: May 27, 2021

(54) SUMMARIZING MEDICATION EVENTS BASED ON MULTIDIMENSIONAL INFORMATION EXTRACTED FROM A DATA SOURCE

(71) Applicant: International Business Machines Corporation, Armonk, NY (US)

(72) Inventors: Diwakar Mahajan, New York, NY (US); Jennifer J. Liang, New York, NY (US); Bharath Dandala, White Plains, NY (US); Ching-Huei Tsou, Briarcliff Manor, NY (US)

(21) Appl. No.: 16/697,579

(22) Filed: Nov. 27, 2019

(51) Int. Cl. G16H 15/00; G16H 10/60; G16H 20/10; G06N 20/00

(52) U.S. Cl. CPC G16H 15/00; G06N 20/00; G16H 20/10; G16H 10/60

(57) ABSTRACT

Systems, computer-implemented methods, and computer program products that can facilitate summarization of medication events based on multidimensional medication event data extracted from a data source are provided. According to an embodiment, a system can comprise a memory that stores computer executable components and a processor that executes the computer executable components stored in the memory. The computer executable components can comprise an extraction component that employs a model to extract multidimensional medication event data of one or more medication events from at least one data source. The computer executable components can further comprise a classification component that classifies the one or more medication events into orthogonal dimensions based on the multidimensional medication event data.

### BACKGROUND

The subject disclosure relates to summarizing medication events, and more specifically, to summarizing medication events based on multidimensional medication event data extracted from a data source.

### SUMMARY

The following presents a summary to provide a basic understanding of one or more embodiments of the invention. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, systems, computer-implemented methods, and/or computer program products that can facilitate summarization of medication events based on multidimensional medication event data extracted from a data source are described.

According to an embodiment, a system can comprise a memory that stores computer executable components and a processor that executes the computer executable components stored in the memory. The computer executable components can comprise an extraction component that employs a model to extract multidimensional medication event data of one or more medication events from at least one data source. The computer executable components can further comprise a classification component that classifies the one or more medication events into orthogonal dimensions based on the multidimensional medication event data. An advantage of such a system is that it can improve the accuracy (e.g., completeness) of a summary of the one or more medication events generated by at least one of the system, the processor, or a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such a system is that it can improve the accuracy (e.g., completeness) of a summary of the one or more medication events generated by at least one of the system, the processor, or a medication reconciliation system.

According to another embodiment, a computer-implemented method can comprise employing, by a system operatively coupled to a processor, a model to extract multidimensional medication event data of one or more medication events from at least one data source. The computer-implemented method can further comprise classifying, by the system, the one or more medication events into orthogonal dimensions based on the multidimensional medication event data. An advantage of such a computer-implemented method is that it can be implemented to improve the accuracy (e.g., completeness) of a summary of the one or more medication events generated by at least one of the system, the processor, or a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such a computer-implemented method is that it can be implemented to improve the accuracy (e.g., completeness) of a summary of the one or more medication events generated by at least one of the system, the processor, or a medication reconciliation system.

According to another embodiment, a computer program product facilitating a process to summarize medication events based on multidimensional medication event data extracted from a data source is provided. The computer program product comprising a computer readable storage medium having program instructions embodied therewith, the program instructions executable by a processor to cause the processor to employ, by the processor, a model to extract multidimensional medication event data of one or more medication events from at least one data source. The program instructions are further executable by the processor to cause the processor to classify, by the processor, the one or more medication events into orthogonal dimensions based on the multidimensional medication event data. An advantage of such a computer program product is that it can improve the accuracy (e.g., completeness) of a summary of the one or more medication events generated by at least one of the processor or a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such a computer program product is that it can improve the accuracy (e.g., completeness) of a summary of the one or more medication events generated by at least one of the processor or a medication reconciliation system.

According to an embodiment, a system can comprise a memory that stores computer executable components and a processor that executes the computer executable components stored in the memory. The computer executable components can comprise an extraction component that employs a model to extract multidimensional medication event data of one or more medication events from at least one data source. The computer executable components can further comprise a summarization component that generates an interactive summary of the medication events based on the multidimensional medication event data. An advantage of such a system is that it can improve the accuracy (e.g., completeness) of a summary of the one or more medication events generated by at least one of the system, the processor, or a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such a system is that it can improve the accuracy (e.g., completeness) of a summary of the one or more medication events generated by at least one of the system, the processor, or a medication reconciliation system.

According to another embodiment, a computer-implemented method can comprise employing, by a system operatively coupled to a processor, a model to extract multidimensional medication event data of one or more medication events from at least one data source. The computer-implemented method can further comprise generating, by the system, an interactive summary of the medication events based on the multidimensional medication event data. An advantage of such a computer-implemented method is that it can be implemented to improve the accuracy (e.g., completeness) of a summary of the one or more medication events generated by at least one of the system, the processor, or a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such a computer-implemented method is that it can be implemented to improve the accuracy (e.g., completeness) of a summary of the one or more medication events generated by at least one of the system, the processor, or a medication reconciliation system.

### DESCRIPTION OF THE DRAWINGS

FIG. 1 illustrates a block diagram of an example, non-limiting system that can facilitate summarization of medication events based on multidimensional medication event data extracted from a data source in accordance with one or more embodiments described herein.

FIG. 2 illustrates a block diagram of an example, non-limiting system that can facilitate summarization of medication events based on multidimensional medication event data extracted from a data source in accordance with one or more embodiments described herein.

FIGS. 3A and 3B illustrate diagrams of example, non-limiting information that can facilitate summarization of medication events based on multidimensional medication event data extracted from a data source in accordance with one or more embodiments described herein.

FIGS. 4A and 4B illustrate block diagrams of example, non-limiting systems that can facilitate summarization of medication events based on multidimensional medication event data extracted from a data source in accordance with one or more embodiments described herein.

FIG. 5 illustrates a diagram of example, non-limiting information that can facilitate summarization of medication events based on multidimensional medication event data extracted from a data source in accordance with one or more embodiments described herein.

FIGS. 6A, 6B, and 6C illustrate diagrams of example, non-limiting displays that can facilitate summarization of medication events based on multidimensional medication event data extracted from a data source in accordance with one or more embodiments described herein.

FIGS. 7, 8, and 9 illustrate flow diagrams of example, non-limiting computer-implemented methods that can facilitate summarization of medication events based on multidimensional medication event data extracted from a data source in accordance with one or more embodiments described herein.

FIG. 10 illustrates a block diagram of an example, non-limiting operating environment in which one or more embodiments described herein can be facilitated.

FIG. 11 illustrates a block diagram of an example, non-limiting cloud computing environment in accordance with one or more embodiments of the subject disclosure.

FIG. 12 illustrates a block diagram of example, non-limiting abstraction model layers in accordance with one or more embodiments of the subject disclosure.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Healthcare providers need a full understanding of a patient's medication history to provide appropriate treatment recommendations. This medication history is captured in various sources of clinical data, including documentation in unstructured clinical notes, structured medication orders, pharmacy prescription filling data, and/or other sources of clinical data. Structured medication orders are more easily accessible in today's electronic health record (EHR) systems, and is usually where healthcare providers go to learn about a patient's medication history. However, many medication events are documented only in unstructured clinical notes and therefore difficult to find and easy to miss, especially at the point-of-care where time is limited.

The following example scenarios cover some use cases where it is useful to extract medication events from unstructured clinical notes. As referenced herein, a medication event can be any documented adjustment—past, present, or future—of a medication for a given patient.

There are a variety of scenarios where medication events may not be reflected in the structured data and only found in clinical notes. This includes both dosage adjustments recommended by the physician as well as those initiated by the patient. A few examples are provided here: a) if the physician has access to sample medications in their office, they may elect to give the patient samples to try taking first before formally putting in a prescription order to the pharmacy; b) if a patient already has a prescription for a medication, the physician may elect to tell the patient to change the dosage (e.g., take 1 pill a day instead of 2 pills) without writing a new prescription since patient already has the medication; c) physician may ask the patient to temporarily stop taking a medication for various reasons, such as to prepare for an upcoming procedure, to trial the patient off medication, or to wait for an abnormal lab result to normalize; d) patient may decide to take medications that were prescribed to a friend or family member; e) patient may be taking a different dosage than prescribed because they misunderstood the instructions given; f) patient may decide to take a different dosage than prescribed to try to manage their problem on their own (e.g., taking a higher dose because patient felt like they needed more to control their symptoms); and/or g) patient may decide to stop taking medication for various reasons, such as adverse effects, ran out of medication, insurance concerns, or financial concerns.

Medication reconciliation is an important use case for medication event extraction from EHRs. However, due to the nature of clinical documentation—which may capture the longitudinal history of a medication in a patient and/or any clinical reasoning behind a physician's treatment decisions—extraction of dosage adjustment actions (e.g., start, increase, etc.) alone is insufficient for the task of medication reconciliation.

To be useful for medication reconciliation, a system should extract not only the dosage adjustment action, but also place it in the context of the documented clinical discussion. A few examples are described here: 1) physician may discuss starting a medication but refrains from doing so because of specific concerns related to the patient (e.g., “we might start with an ace-inhibitor but given the history of side effects (s.e.) I'm reluctant”)—indicates an action that was considered but not implemented, not useful for reconciliation; 2) physician documents a change in problem status as a result of a medication which was stopped in the past (e.g., “blood pressure (BP) stable off lisinopril”)—indicates past stop action, maybe useful for reconciliation; 3) physician describes a dosage adjustment action contingent on a stated condition (e.g., “if there is an elevation in chromium (Cr) we will decrease Lasix to 40 milligrams (mg) every morning (quaque ante meridiem (qam)) and 20 mg every afternoon or evening (quaque post meridiem (qpm))”)—indicates a potential future action that may occur depending on whether condition is met, maybe useful for reconciliation; and/or 4) patient-initiated dosage adjustment action (e.g., “she was experiencing a bad episode of dry cough so she stopped taking lisinopril”)—indicates past action taken by patient between clinical visits, useful for reconciliation.

Some existing medication events summarization technologies identify medication status (e.g., medication actions such as, for instance, start, stop, increase, decrease, no change, etc.) from clinical text. However, a problem with such technologies is that they fail to address issues of various dimensions such as, for instance: temporality (e.g., whether this change happened in the past, present, or future); certainty (e.g., whether this change was hypothetical or certain to have happened); and/or the actor who initiated the change (e.g., physician, patient, etc.). Thus, such technologies fail to extract the complete picture of medication events from EHR. For example, while such technologies can extract medication dosage adjustment actions from clinical notes, they fail to identify various aspects of the actions such as, for instance: the temporality (e.g., past, present, future, etc.); the certainty (e.g., conditional, hypothetical, actually happened, etc.); and/or the actor (e.g., physician, patient, etc.).

Another problem with some existing medication events summarization technologies is that they do not extract medication event information from both structured and unstructured data within the patient electronic health record (EHR). Another problem with some existing medication events summarization technologies is that they do not provide an interactive summary of medication events that can be organized in chronological order and/or filtered based on one or more attributes of data corresponding to the medication events.

Given the problems described above with existing technologies failing to extract multidimensional medication events data (e.g., action, temporality, certainty, actor, etc.) from both structured and unstructured data, the present disclosure can be implemented to produce a solution to this problem in the form of systems, computer-implemented methods, and/or computer program products that can employ a model to extract multidimensional medication event data (e.g., action, temporality, certainty, actor, etc.) of one or more medication events from at least one data source; and/or classify the one or more medication events into orthogonal dimensions based on the multidimensional medication event data. An advantage of such systems, computer-implemented methods, and/or computer program products is that they can improve the accuracy (e.g., completeness) of a summary of the one or more medication events generated by, for instance, a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such systems, computer-implemented methods, and/or computer program products is that they can improve the accuracy (e.g., completeness) of a summary of the one or more medication events generated by, for instance, a medication reconciliation system.

Given the problem described above with existing technologies failing to provide an interactive summary of medication events that can be organized in chronological order and/or filtered based on one or more attributes of data corresponding to the medication events, the present disclosure can be implemented to produce a solution to this problem in the form of systems, computer-implemented methods, and/or computer program products that can employ a model to extract multidimensional medication event data (e.g., action, temporality, certainty, actor, etc.) of medication events from at least one data source; and/or generate an interactive summary of the medication events based on the multidimensional medication event data. An advantage of such systems, computer-implemented methods, and/or computer program products is that they can improve the accuracy (e.g., completeness) of a summary of the one or more medication events generated by, for instance, a medication reconciliation system.

In some embodiments, the at least one data source is selected from a group consisting of an electronic health record, an electronic medical record, a structured data source, a structured medication order, an unstructured data source, an unstructured clinical note, and pharmacy prescription filling data. An advantage of such systems, computer-implemented methods, and/or computer program products is that they can improve the accuracy (e.g., completeness) of a summary of the one or more medication events generated by, for instance, a medication reconciliation system.

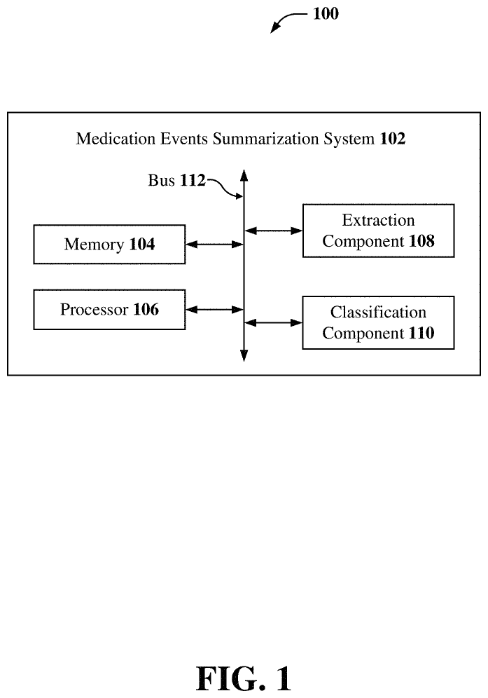

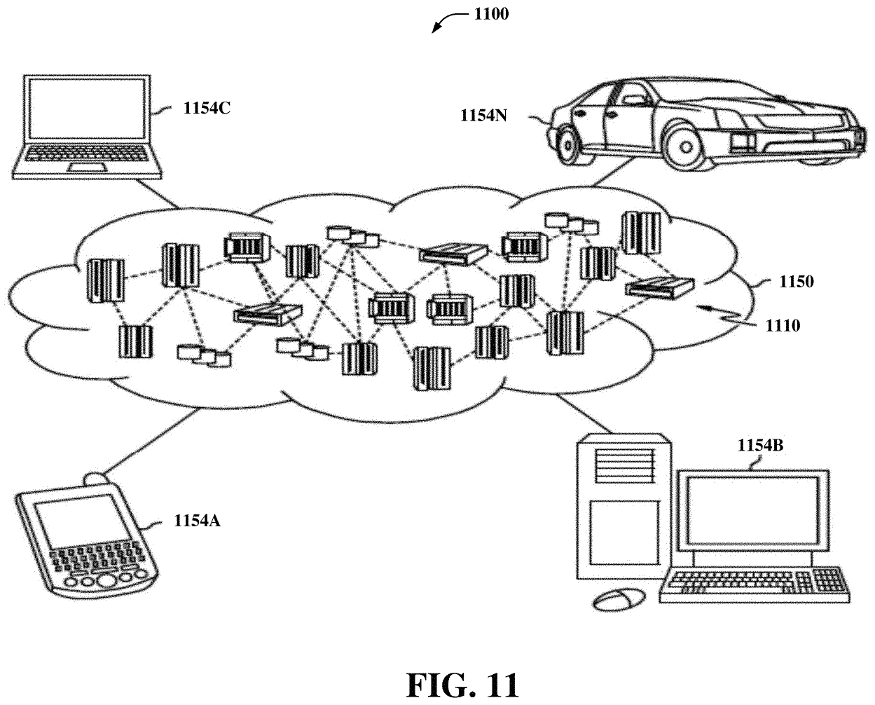

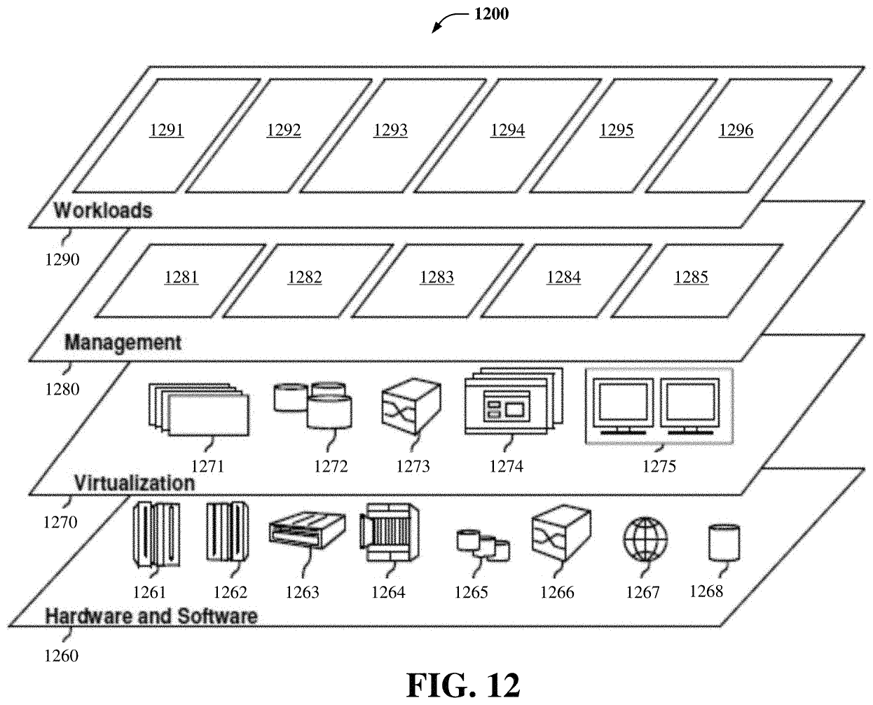

FIG. 1 illustrates a block diagram of an example, non-limiting system 100 that can facilitate summarization of medication events based on multidimensional medication event data extracted from a data source in accordance with one or more embodiments described herein. In some embodiments, system 100 can comprise a medication events summarization system 102, which can be associated with a cloud computing environment. For example, medication events summarization system 102 can be associated with cloud computing environment 1150 described below with reference to FIG. 11 and/or one or more functional abstraction layers described below with reference to FIG. 12 (e.g., hardware and software layer 1260, virtualization layer 1270, management layer 1280, and/or workloads layer 1290).

In some embodiments, medication events summarization system 102 and/or components thereof (e.g., extraction component 108, classification component 110, summarization component 202, filter component 204, etc.) can employ one or more computing resources of cloud computing environment 1150 described below with reference to FIG. 11 and/or one or more functional abstraction layers described below with reference to FIG. 12 to execute one or more operations in accordance with one or more embodiments of the subject disclosure described herein. For example, cloud computing environment 1150 and/or such one or more functional abstraction layers can comprise one or more classical computing devices (e.g., classical computer, classical processor, virtual machine, server, etc.) and/or one or more quantum computing devices (e.g., quantum computer, quantum processor, quantum circuit simulation software, superconducting circuit, etc.) that can be employed by medication events summarization system 102 and/or components thereof to execute one or more operations in accordance with one or more embodiments of the subject disclosure described herein. For instance, medication events summarization system 102 and/or components thereof can employ such one or more classical and/or quantum computing devices to execute one or more mathematical functions and/or equations, one or more computing and/or processing scripts, one or more models (e.g., artificial intelligence (AI) models, machine learning (ML) models, etc.), one or more classical and/or quantum algorithms, and/or another operation in accordance with one or more embodiments of the subject disclosure described herein.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Continuing now with FIG. 1. According to several embodiments, medication events summarization system 102 can comprise a memory 104, a processor 106, an extraction component 108, a classification component 110, and/or a bus 112.

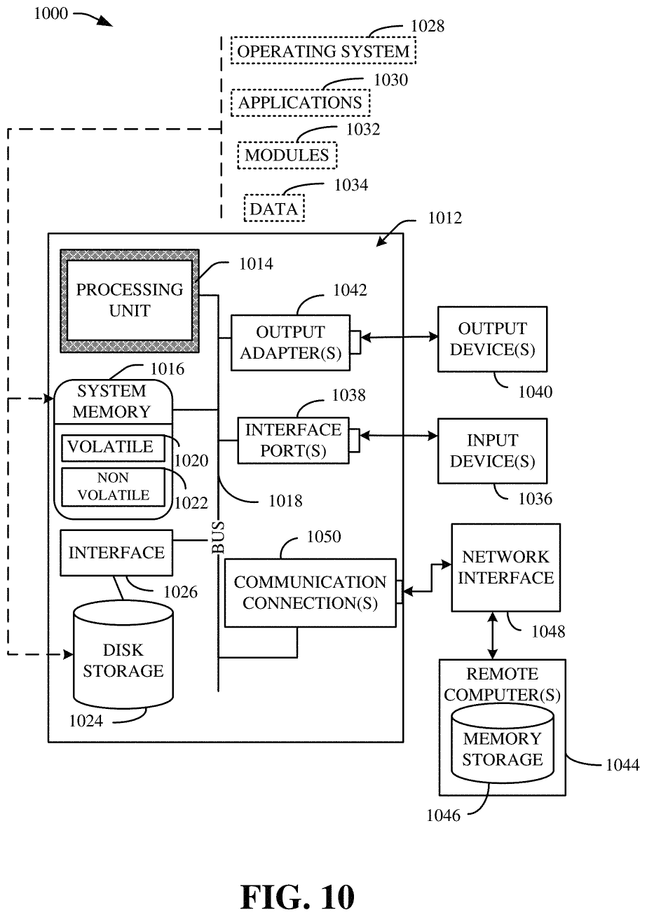

It should be appreciated that the embodiments of the subject disclosure depicted in various figures disclosed herein are for illustration only, and as such, the architecture of such embodiments are not limited to the systems, devices, and/or components depicted therein. For example, in some embodiments, system 100 and/or medication events summarization system 102 can further comprise various computer and/or computing-based elements described herein with reference to operating environment 1000 and FIG. 10. In several embodiments, such computer and/or computing-based elements can be used in connection with implementing one or more of the systems, devices, components, and/or computer-implemented operations shown and described in connection with FIG. 1 or other figures disclosed herein.

Memory 104 can store one or more computer and/or machine readable, writable, and/or executable components and/or instructions that, when executed by processor 106 (e.g., a classical processor, a quantum processor, etc.), can facilitate performance of operations defined by the executable component(s) and/or instruction(s). For example, memory 104 can store computer and/or machine readable, writable, and/or executable components and/or instructions that, when executed by processor 106, can facilitate execution of the various functions described herein relating to medication events summarization system 102, extraction component 108, classification component 110, and/or another component associated with medication events summarization system 102 (e.g., summarization component 202, filter component 204, etc.), as described herein with or without reference to the various figures of the subject disclosure.

Memory 104 can comprise volatile memory (e.g., random access memory (RAM), static RAM (SRAM), dynamic RAM (DRAM), etc.) and/or non-volatile memory (e.g., read only memory (ROM), programmable ROM (PROM), electrically programmable ROM (EPROM), electrically erasable programmable ROM (EEPROM), etc.) that can employ one or more memory architectures. Further examples of memory 104 are described below with reference to system memory 1016 and FIG. 10. Such examples of memory 104 can be employed to implement any embodiments of the subject disclosure.

Processor 106 can comprise one or more types of processors and/or electronic circuitry (e.g., a classical processor, a quantum processor, etc.) that can implement one or more computer and/or machine readable, writable, and/or executable components and/or instructions that can be stored on memory 104. For example, processor 106 can perform various operations that can be specified by such computer and/or machine readable, writable, and/or executable components and/or instructions including, but not limited to, logic, control, input/output (I/O), arithmetic, and/or the like. In some embodiments, processor 106 can comprise one or more central processing unit, multi-core processor, microprocessor, dual microprocessors, microcontroller, System on a Chip (SOC), array processor, vector processor, quantum processor, and/or another type of processor. Further examples of processor 106 are described below with reference to processing unit 1014 and FIG. 10. Such examples of processor 106 can be employed to implement any embodiments of the subject disclosure.

Medication events summarization system 102, memory 104, processor 106, extraction component 108, classification component 110, and/or another component of medication events summarization system 102 as described herein can be communicatively, electrically, operatively, and/or optically coupled to one another via a bus 112 to perform functions of system 100, medication events summarization system 102, and/or any components coupled therewith. In several embodiments, bus 112 can comprise one or more memory bus, memory controller, peripheral bus, external bus, local bus, a quantum bus, and/or another type of bus that can employ various bus architectures. Further examples of bus 112 are described below with reference to system bus 1018 and FIG. 10. Such examples of bus 112 can be employed to implement any embodiments of the subject disclosure.

Medication events summarization system 102 can comprise any type of component, machine, device, facility, apparatus, and/or instrument that comprises a processor and/or can be capable of effective and/or operative communication with a wired and/or wireless network. All such embodiments are envisioned. For example, medication events summarization system 102 can comprise a server device, a computing device, a general-purpose computer, a special-purpose computer, a quantum computing device (e.g., a quantum computer), a tablet computing device, a handheld device, a server class computing machine and/or database, a laptop computer, a notebook computer, a desktop computer, a cell phone, a smart phone, a consumer appliance and/or instrumentation, an industrial and/or commercial device, a digital assistant, a multimedia Internet enabled phone, a multimedia players, and/or another type of device.

Medication events summarization system 102 can be coupled (e.g., communicatively, electrically, operatively, optically, etc.) to one or more external systems, sources, and/or devices (e.g., classical and/or quantum computing devices, communication devices, etc.) via a data cable (e.g., High-Definition Multimedia Interface (HDMI), recommended standard (RS) 232, Ethernet cable, etc.). In some embodiments, medication events summarization system 102 can be coupled (e.g., communicatively, electrically, operatively, optically, etc.) to one or more external systems, sources, and/or devices (e.g., classical and/or quantum computing devices, communication devices, etc.) via a network.

In some embodiments, such a network can comprise wired and wireless networks, including, but not limited to, a cellular network, a wide area network (WAN) (e.g., the Internet) or a local area network (LAN). For example, medication events summarization system 102 can communicate with one or more external systems, sources, and/or devices, for instance, computing devices (and vice versa) using virtually any desired wired or wireless technology, including but not limited to: wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (IPv6 over Low power Wireless Area Networks), Z-Wave, an ANT, an ultra-wideband (UWB) standard protocol, and/or other proprietary and non-proprietary communication protocols. In such an example, medication events summarization system 102 can thus include hardware (e.g., a central processing unit (CPU), a transceiver, a decoder, a quantum processor, etc.), software (e.g., a set of threads, a set of processes, software in execution, quantum pulse schedule, quantum circuit, etc.) or a combination of hardware and software that facilitates communicating information between medication events summarization system 102 and external systems, sources, and/or devices (e.g., computing devices, communication devices, etc.).

Medication events summarization system 102 can comprise one or more computer and/or machine readable, writable, and/or executable components and/or instructions that, when executed by processor 106 (e.g., a classical processor, a quantum processor, etc.), can facilitate performance of operations defined by such component(s) and/or instruction(s). Further, in numerous embodiments, any component associated with medication events summarization system 102, as described herein with or without reference to the various figures of the subject disclosure, can comprise one or more computer and/or machine readable, writable, and/or executable components and/or instructions that, when executed by processor 106, can facilitate performance of operations defined by such component(s) and/or instruction(s). For example, extraction component 108, classification component 110, and/or any other components associated with medication events summarization system 102 as disclosed herein (e.g., communicatively, electronically, operatively, and/or optically coupled with and/or employed by medication events summarization system 102), can comprise such computer and/or machine readable, writable, and/or executable component(s) and/or instruction(s). Consequently, according to numerous embodiments, medication events summarization system 102 and/or any components associated therewith as disclosed herein, can employ processor 106 to execute such computer and/or machine readable, writable, and/or executable component(s) and/or instruction(s) to facilitate performance of one or more operations described herein with reference to medication events summarization system 102 and/or any such components associated therewith.

Medication events summarization system 102 can facilitate performance of operations executed by and/or associated with extraction component 108, classification component 110, and/or another component associated with medication events summarization system 102 as disclosed herein (e.g., summarization component 202, filter component 204, etc.). For example, as described in detail below, medication events summarization system 102 can facilitate via processor 106 (e.g., a classical processor, a quantum processor, etc.): employing a model to extract multidimensional medication event data of one or more medication events from at least one data source; and/or classifying the one or more medication events into orthogonal dimensions based on the multidimensional medication event data. In another example, medication events summarization system 102 can further facilitate via processor 106 (e.g., a classical processor, a quantum processor, etc.): generating a summary of the one or more medication events based on the multidimensional medication event data; and/or filtering at least one of a summary of the one or more medication events, the multidimensional medication event data, the one or more medication events, or the at least one data source based on at least one orthogonal dimension of the multidimensional medication event data. In some embodiments: the at least one data source can comprise an electronic health record, an electronic medical record, a structured data source, a structured medication order, an unstructured data source, an unstructured clinical note, and/or pharmacy prescription filling data; and/or the orthogonal dimensions can comprise action, temporality, certainty, and/or actor.

In another example, medication events summarization system 102 can further facilitate via processor 106 (e.g., a classical processor, a quantum processor, etc.): employing a model to extract multidimensional medication event data of medication events from at least one data source; and/or generating an interactive summary of the medication events based on the multidimensional medication event data. In another example, medication events summarization system 102 can further facilitate via processor 106 (e.g., a classical processor, a quantum processor, etc.): determining daily medication dosage data based on the multidimensional medication event data; classifying the medication events into orthogonal dimensions based on the multidimensional medication event data, wherein the orthogonal dimensions are selected from a group consisting of action, temporality, certainty, and actor; and/or filtering at least one of the interactive summary, the multidimensional medication event data, the medication events, or the at least one data source based on at least one orthogonal dimension of the multidimensional medication event data. In some embodiments, the at least one data source can comprise an electronic health record, an electronic medical record, a structured data source, a structured medication order, an unstructured data source, an unstructured clinical note, and/or pharmacy prescription filling data.

Extraction component 108 can employ a model to extract multidimensional medication event data of one or more medication events from at least one data source. For example, to facilitate such extraction, extraction component 108 can employ a machine learning (ML) model based on Artificial Intelligence (AI) and Natural Language Processing (NLP), including, but not limited to, a long short-term memory (LSTM) model, a bidirectional LSTM model with a conditional random field (CRF) layer (abbreviated as BiLSTM-CRF), a pretrained language model (e.g., transformer based) fine-tuning, a shallow or deep neural network model, a convolutional neural network (CNN) model, a support vector machine (SVM) model, a decision tree classifier, and/or any supervised or unsupervised machine learning model.

Extraction component 108 can employ one or more models defined above to extract multidimensional medication event data corresponding to one or more past, present, or future medication events that can comprise a change in medication status. For example, extraction component 108 can employ one or more of such models defined above to extract multidimensional medication event data including, but not limited to, medication name, medication type, medication formulation (e.g., pill, liquid, etc.), medication artifacts, medication sig (e.g., instructions on how to take the medication), medication dosage, medication strength, patient name, medication provider name, and/or other multidimensional medication event data.

Extraction component 108 can employ one or more of such models defined above to extract multidimensional medication event data corresponding to one or more medication events from one or more data sources, where such data source(s) can comprise structured and/or unstructured data sources. For example, extraction component 108 can employ one or more of such models defined above to extract multidimensional medication event data corresponding to one or more medication events from one or more data sources including, but not limited to an electronic health record (EHR), an electronic medical record (EMR), a structured medication order, an unstructured clinical note, pharmacy prescription filling data, and/or another data source.

Classification component 110 can classify one or more medication events into orthogonal dimensions based on multidimensional medication event data. For example, classification component 110 can classify one or more past, present, or future medication events that can comprise a change in medication status by classifying multidimensional medication event data corresponding to such one or more medication events into one or more orthogonal dimensions. For instance, classification component 110 can classify multidimensional medication event data corresponding to such one or more medication events into one or more orthogonal dimensions including, but not limited to, action, temporality, certainty, actor, and/or another orthogonal dimension. In an example, for each medication event mentioned in a data source (e.g., an unstructured clinical note), classification component 110 can assign values to the event based on multiple dimensions (e.g., action, temporality, certainty, actor, etc.) that are orthogonal to each other (e.g., meaning the value of each aspect is independent of any other aspect).

To facilitate such classification, classification component 110 can employ one or more of the models defined above. For instance, classification component 110 can employ a classification model (e.g., a logistic regression model, a naïve Bayes model, a stochastic gradient descent model, a k-nearest neighbors model, a decision tree model, a random forest model, a support vector machine (SVM) model, etc.) to classify one or more medication events into orthogonal dimensions based on multidimensional medication event data.

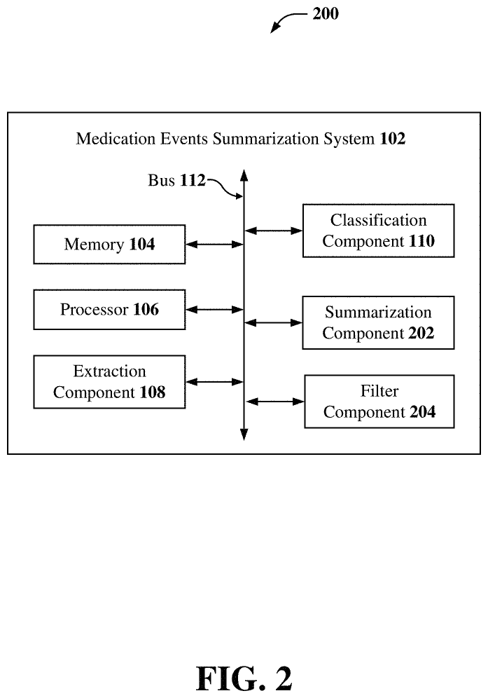

It should be appreciated that classification component 110 can classify medication events into orthogonal dimensions based on multidimensional medication event data as described above to improve accuracy (e.g., completeness) of a summary (e.g., an interactive summary) of the medication events. It should be further appreciated that classification component 110 can classify medication events into orthogonal dimensions based on multidimensional medication event data as described above to improve performance of a component, device, and/or system that can generate such a summary (e.g., medication events summarization system 102, processor 106, summarization component 202 described herein with reference to FIG. 2, a medication reconciliation system implementing medication events summarization system 102, etc.).

FIG. 2 illustrates a block diagram of an example, non-limiting system 200 that can facilitate summarization of medication events based on multidimensional medication event data extracted from a data source in accordance with one or more embodiments described herein. In some embodiments, system 200 can comprise medication events summarization system 102. In some embodiments, medication events summarization system 102 can comprise a summarization component 202 and/or a filter component 204. Repetitive description of like elements and/or processes employed in respective embodiments is omitted for sake of brevity.

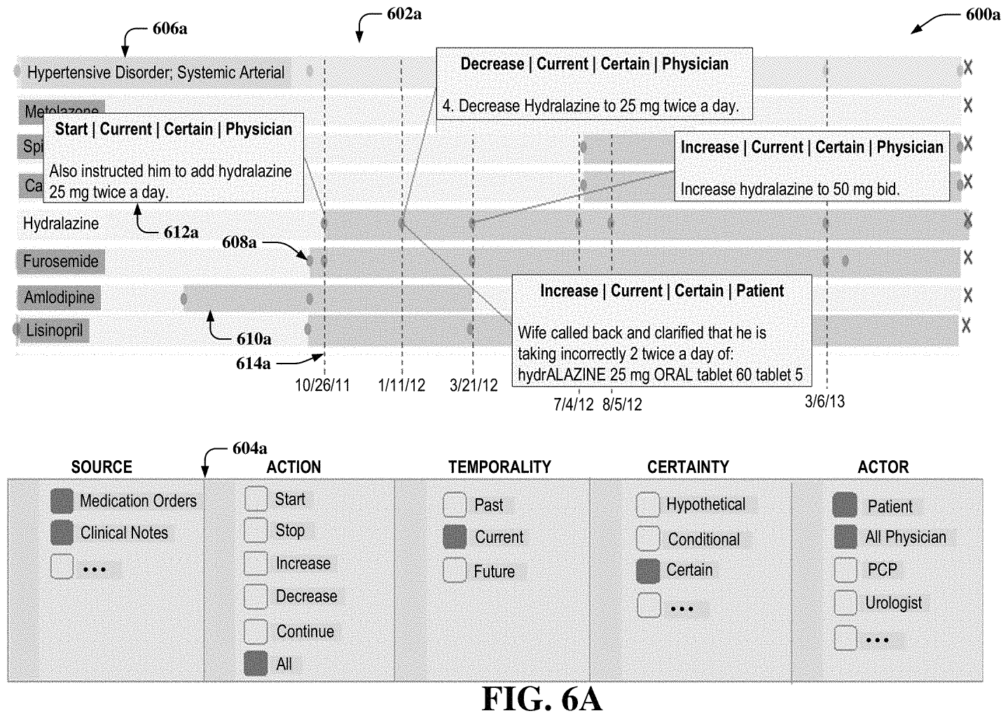

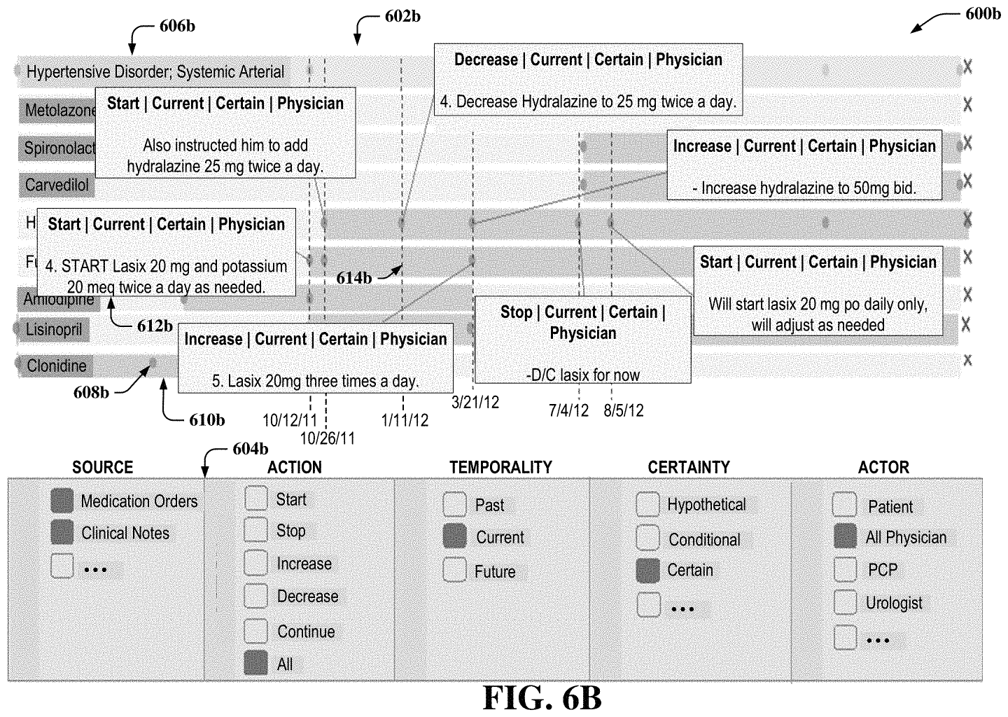

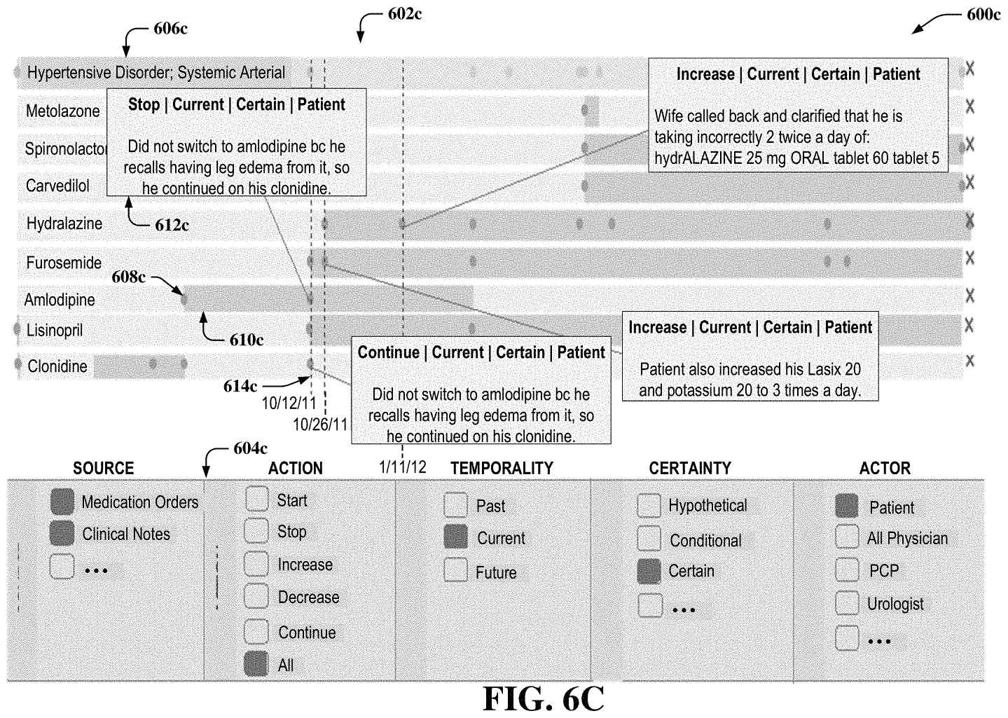

Summarization component 202 can generate a summary of one or more medication events based on multidimensional medication event data. For example, based on multidimensional medication event data corresponding to one or more medication events that can be extracted by extraction component 108 and/or classified by classification component 110 as described above, summarization component 202 can generate a summary of such medication event(s). In an example, summarization component 202 can generate a chronological summary of multiple medication events plotted on a timeline. For instance, summarization component 202 can generate summary 602a, 602b, and/or 602c described below and illustrated in FIGS. 6A, 6B, and 6C, respectively, where each of such summaries can comprise a chronological summary of multiple medication events plotted on a timeline.

Summarization component 202 can further determine daily medication dosage data based on multidimensional medication event data. For example, based on multidimensional medication event data that can be extracted by extraction component 108 such as, for instance, dose per administration, frequency, medication strength, and/or other data, summarization component 202 can employ a set of rules (e.g., a rule-based system) to normalize these extracted values to a daily value (e.g., 2 tablets of 50 mg per day).

In some embodiments, summarization component 202 can generate an interactive summary of one or more medication events based on multidimensional medication event data. For example, based on multidimensional medication event data corresponding to one or more medication events that can be extracted by extraction component 108 and/or classified by classification component 110 as described above, summarization component 202 can generate an interactive summary of such medication event(s). For instance, summarization component 202 can generate summary 602a, 602b, and/or 602c described below and illustrated in FIGS. 6A, 6B, and 6C, respectively, where each of such summaries can comprise an interactive summary that can be filtered (e.g., via filter component 204) to display certain information corresponding to the medication event(s).

Filter component 204 can filter a summary (e.g., an interactive summary) of one or more medication events, multidimensional medication event data corresponding to such medication event(s), the one or more medication events themselves, and/or a data source based on at least one orthogonal dimension of the multidimensional medication event data. For example, with reference to FIGS. 6A, 6B, 6C, filter component 204 can filter summary 602a, 602b, and/or 602c by filtering the multidimensional medication event data of such summaries, the one or more medication events themselves, and/or a data source from which the multidimensional medication event data was extracted (e.g., via extraction component 108) based on at least one orthogonal dimension (e.g., action, temporality, certainty, actor, etc.) of the multidimensional medication event data.

In an example, an entity can utilize an interface component of medication events summarization system 102 (e.g., an application programming interface (API), a representational state transfer API, a graphical user interface (GUI), etc.) to employ filter component 204 to filter a summary (e.g., an interactive summary such as, for instance, summary 602a, 602b, 602c of FIGS. 6A, 6B, 6C, respectively) of one or more medication events as described above. In several embodiments, such an entity can include, but not limited to, a human, a client, a user, a computing device, a software application, an agent, a machine learning (ML) model, an artificial intelligence (AI) model, and/or another entity.

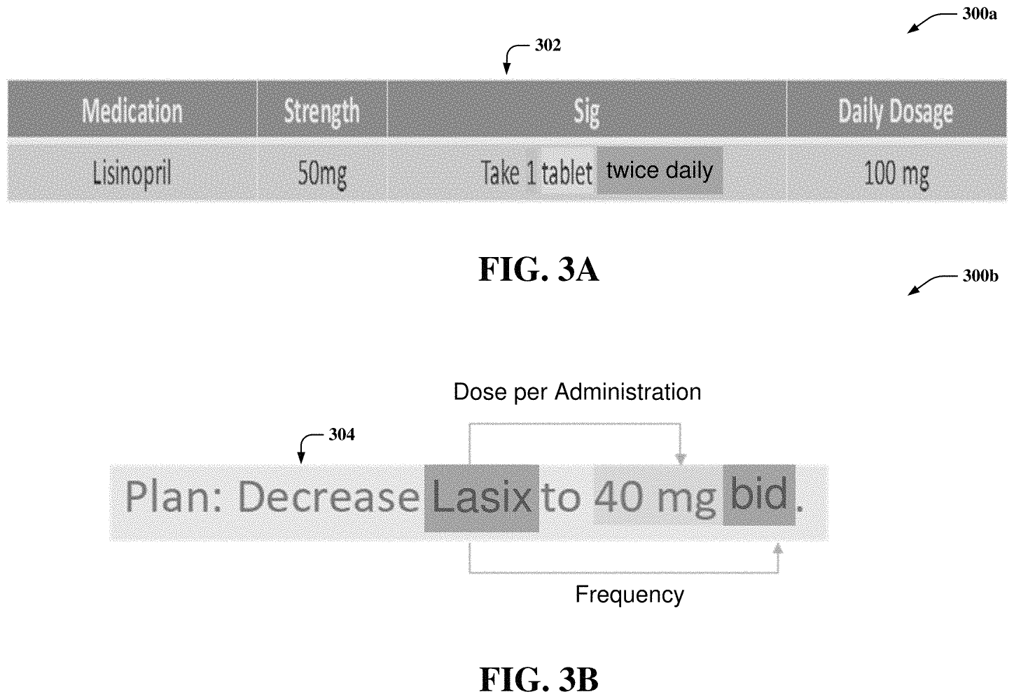

FIGS. 3A and 3B illustrate diagrams of example, non-limiting information 300a, 300b that can facilitate summarization of medication events based on multidimensional medication event data extracted from a data source in accordance with one or more embodiments described herein. Repetitive description of like elements and/or processes employed in respective embodiments is omitted for sake of brevity.

Information 300a illustrated in FIG. 3A can comprise a table 302 that can be generated by medication events summarization system 102 (e.g., via extraction component 108, classification component 110, and/or summarization component 202). In an example, extraction component 108 can employ one or more of such models defined above to extract multidimensional medication event data corresponding to one or more medication events from a medication order in an EHR's structured data which comprises the medication name, strength, and sig (e.g., instructions on how to take the medication). For instance, extraction component 108 can employ one or more of such models defined above (e.g., a BiLSTM-CRF model, a pretrained language model (e.g., transformer based) fine-tuning, etc.) to extract the dose per administration, frequency, route, duration, and/or formulation from sig. This information, when combined with strength, allows medication events summarization system 102 to calculate the daily dosage (e.g., via summarization component 202 as described herein with reference to FIG. 2).

For example, with reference to FIG. 3A, the columns of table 302 denoted as Medication, Strength, and Sig can be a part of a structured data source (e.g., a Medication Order in an HER). The Sig column can be analyzed by extraction component 108 (e.g., using one or more models defined above) to extract: dosage-per-administration (denoted as “1” in FIG. 3A); frequency (denoted as “twice daily” in FIG. 3A); and/or formulation (denoted as “tablet” in FIG. 3A). In some embodiments, medication events summarization system 102 (e.g., via summarization component 202) can employ a set of rules (e.g., a rule-based system) to normalize these extracted values to a daily value (e.g., 2 tablets per day), which when combined with Strength (denoted as “50 mg” in FIG. 3A) can yield the fourth column of table 302 denoted as Daily Dosage in FIG. 3A for the medication (denoted as “100 mg” in FIG. 3A).

Information 300b illustrated in FIG. 3B can comprise an excerpt 304 from a clinical note. As described above with reference to FIG. 1, extraction component 108 can employ one or more models defined above to identify a medication and/or its related artifacts (e.g., strength, dose per administration, frequency, route, duration, etc.). This information allows medication events summarization system 102 to calculate the daily dosage (e.g., via summarization component 202 as described herein with reference to FIG. 2).

In an example, extraction component 108 can employ one or more models defined above (e.g., a pretrained language model (e.g., transformer based) fine-tuning, etc.) to analyze the text of excerpt 304 to: (1) extract medication (denoted as “Lasix” in FIG. 3B), dose per administration (denoted as “40 mg” in FIG. 3B), and/or frequency (denoted as “bid” in FIG. 3B which can denote “twice a day”); and/or (2) associate these extracted artifacts with the corresponding medication. In some embodiments, medication events summarization system 102 (e.g., via summarization component 202) can employ a set of rules (e.g., a rule-based system) to normalize these extracted values to a daily dosage value (e.g., “80 mg per day”) for the medication.

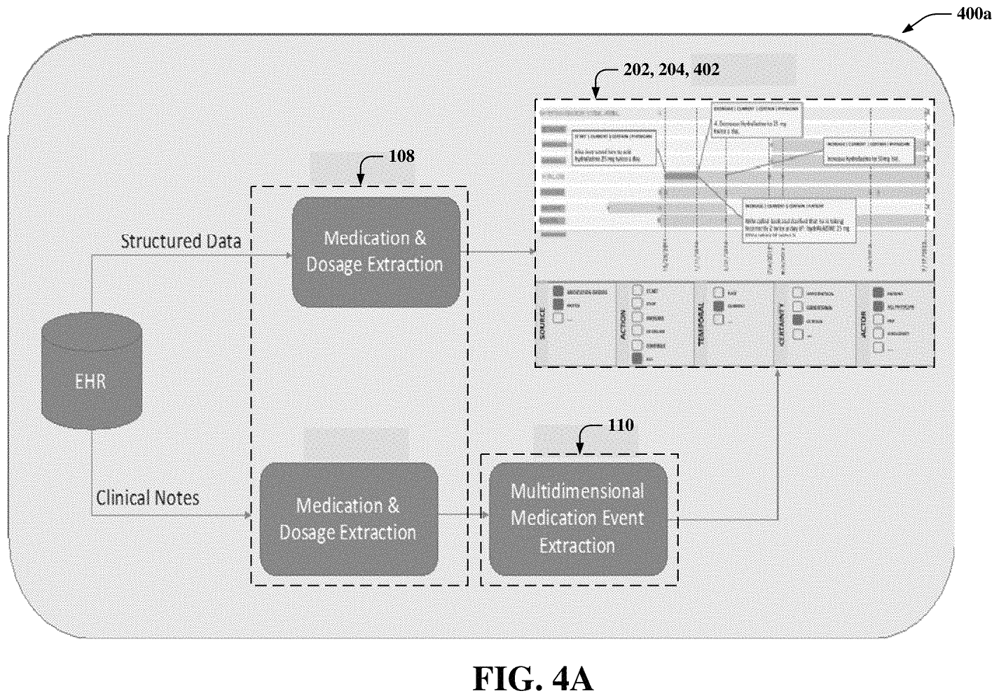

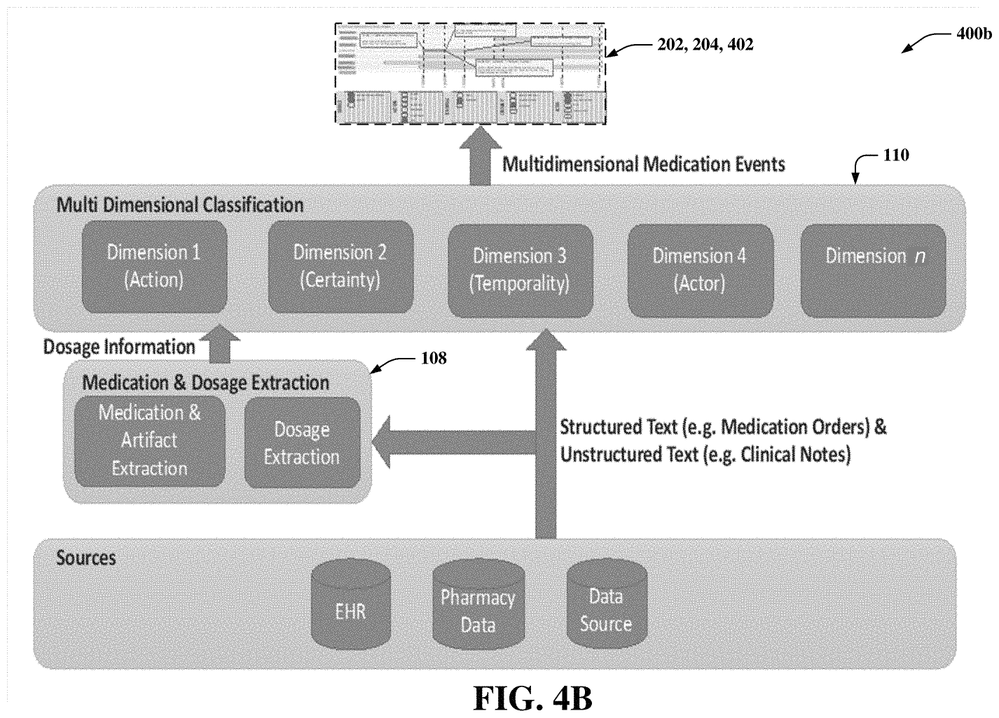

FIGS. 4A and 4B illustrate block diagrams of example, non-limiting systems 400a, 400b that can facilitate summarization of medication events based on multidimensional medication event data extracted from a data source in accordance with one or more embodiments described herein. Repetitive description of like elements and/or processes employed in respective embodiments is omitted for sake of brevity.

As illustrated in FIGS. 4A and 4B, the input to medication events summarization system 102 (e.g., input to extraction component 108 and/or classification component 110) can be medication information corresponding to an entity (e.g., a human patient) that can be obtained from one or more data sources. For example, the input to medication events summarization system 102 (e.g., input to extraction component 108 and/or classification component 110) can be an electronic health record (EHR) of an entity (e.g., a human patient), pharmacy data corresponding to the entity, and/or medication information corresponding to the entity that can be obtained from another data source. In some embodiments, as illustrated in FIGS. 4A and 4B, extraction component 108 and/or classification component 110 can receive as input (e.g., via an interface of medication events summarization system 102 such as, for instance, an API, a REST API, a GUI, etc.) structured data (e.g., structured text comprising a medication order(s)) and/or unstructured data (e.g., unstructured text comprising a clinical note(s)).

Based on receipt of such structured and/or unstructured data, in an example, extraction component 108 can employ one or more of the models defined above with reference to FIG. 1 to analyze structured medication orders and to extract each medication, as well as its associated artifacts and dosage information. In another example, extraction component 108 can employ one or more of the models defined above with reference to FIG. 1 to analyze unstructured clinical notes and extract any medications, as well as their associated artifacts and dosage information.

As illustrated in FIG. 4B, the medication information that can be extracted by extraction component 108 from unstructured clinical notes can be classified across various dimensions (e.g., action, temporality, certainty, actor, etc.) by classification component 110. For instance, classification component 110 can employ one or more of the models defined above with reference to FIG. 1 to classify such medication information into one or more orthogonal dimensions including, but not limited to, action (denoted as Dimension 1 in FIG. 4B), certainty (denoted as Dimension 2 in FIG. 4B), temporality (denoted as Dimension 3 in FIG. 4B), actor (denoted as Dimension 4 in FIG. 4B), and/or another orthogonal dimension (denoted as Dimension n in FIG. 4B).

Based on such medication event information that can be extracted by extraction component 108 from structured and/or unstructured data, summarization component 202 can generate a summary 402 of the medication events and/or the extracted medication event information as illustrated in FIGS. 4A and 4B. For purposes of clarity in the figures, summary 402 depicted in FIGS. 4A and 4B is not intended to be legible, however, summary 402 can comprise an example, non-limiting alternative embodiment of summary 602a, 602b, and/or 602c described below and illustrated in FIGS. 6A, 6B, and 6C, respectively.

Summary 402 can comprise an interactive summary that can be filtered based on one or more attributes of summary 402 and/or one or more attributes of the information in summary 402. For example, filter component 204 can be employed to filter summary 402 based on one or more dimensions (e.g., attributes) of the medication event information (e.g., multidimensional medication event data) that can be extracted from a structured and/or unstructured data source by extraction component 108. For instance, filter component 204 can be employed to filter summary 402 based on one or more orthogonal dimensions corresponding to the medication event information including, but not limited to, the data source from which the medication event information was extracted (e.g., medication orders, clinical notes, EHR, pharmacy data, etc.), the medication name, artifacts of the medication, Dimensions 1, 2, 3, 4, and/or n depicted in FIG. 4B, and/or another dimension.

As described above with reference to FIG. 2, an entity (e.g., a human, a client, a user, a computing device, a software application, an agent, a machine learning (ML) model, an artificial intelligence (AI) model, etc.) can utilize an interface of medication events summarization system 102 (e.g., an API, a REST API, a GUI, etc.) to filter summary 402 by filtering the multidimensional medication event data, the one or more medication events, and/or the data source based on at least one orthogonal dimension (e.g., Dimensions 1, 2, 3, 4, n depicted in FIG. 4B) of the multidimensional medication event data. In these embodiments, such filtration of summary 402 can reduce information overload (e.g., information overload to the entity defined above) and/or can reduce the processing workload of processor 106 in executing summarization requests (e.g., summary operations, instructions, processing threads, etc.) received from summarization component 202.

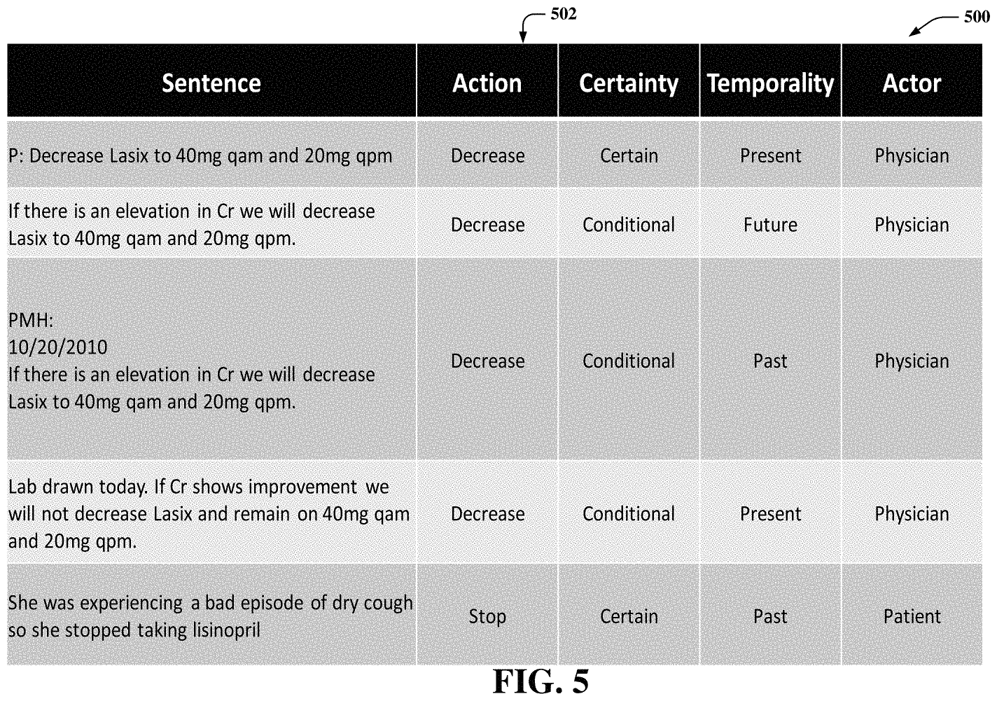

FIG. 5 illustrates a diagram of example, non-limiting information 500 that can facilitate summarization of medication events based on multidimensional medication event data extracted from a data source in accordance with one or more embodiments described herein. Repetitive description of like elements and/or processes employed in respective embodiments is omitted for sake of brevity.

Information 500 can comprise a table 502 that can be generated by medication events summarization system 102 (e.g., via extraction component 108, classification component 110, and/or summarization component 202). As described above with reference to FIG. 1, extraction component 108 can employ one or more models defined above (e.g., a BiLSTM-CRF model, a pretrained language model (e.g., transformer based) fine-tuning, etc.) to identify multidimensional assertions on every medication event mentioned in a clinical note to extract various aspects of the event. Additionally, or alternatively, classification component 110 can employ one or more models defined above (e.g., one or more classification models) to classify the medication event mentioned in the clinical note across various orthogonal dimensions.

In an example, medication events summarization system 102 (e.g., via extraction component 108, classification component 110, and/or summarization component 202) can generate table 502 based on such extraction and/or classification operations described above. For instance, a medication event from unstructured clinical notes can comprise a sentence that extraction component 108 can extract and note in the Sentence column of table 502. In this example, such a medication event can have multidimensional information associated to it that can also be extracted by extraction component 108 such as, for example, action, certainty, temporality, and/or actor. In this example, such multidimensional information can comprise orthogonal dimensions that can be represented by the Action, Certainty, Temporality, and/or Actor columns of table 502 as illustrated in FIG. 5. In this example, classification component 110 can classify the medication event across such orthogonal dimensions based on the multidimensional information. For instance, classification component 110 can classify the medication event noted in the Sentence column of table 502 by assigning a value to each of the orthogonal dimensions of the multidimensional information represented by the Action, Certainty, Temporality, and Actor columns of table 502 illustrated in FIG. 5.

The Action column of table 502 can represent an orthogonal dimension comprising, for example, action values to start, stop, increase, decrease, and/or continue a medication. Classification component 110 can assign any one of such action values (e.g., to start, stop, increase, decrease, and/or continue a medication) to the Action column (e.g., to the Action dimension), where a lack of at least one of such action values in the data source means there is no medication event (e.g., no disposition).

The Temporality column of table 502 can represent an orthogonal dimension comprising, for example, temporality values indicating that the action can be: (1) prescribed in the past and re-stated; (2) prescribed at the current or present visit; or (3) intended for a future date/time. Classification component 110 can assign any one of such temporality values (e.g., past, current, present, or future) to the Temporality column (e.g., to the Temporality dimension).

The Certainty column of table 502 can represent an orthogonal dimension comprising, for example, certainty values indicating that the action can be: (1) something considered by the physician but not yet decided upon (e.g., hypothetical); (2) dependent upon a condition being met (e.g., conditional); or (3) a definitive decision by the physician and/or patient to take an action (e.g., certain). Classification component 110 can assign any one of such certainty values (e.g., hypothetical, conditional, or certain) to the Certainty column (e.g., to the Certainty dimension).

The Actor column of table 502 can represent an orthogonal dimension comprising, for example, actor values indicating that the action can be: (1) instructed by the physician; or (2) initiated by the patient without consulting the physician (e.g., patient nonadherent to medication). Classification component 110 can assign any one of such actor values (e.g., physician or patient) to the Actor column (e.g., to the Actor dimension).

For each medication event mentioned in an unstructured clinical note, classification component 110 can assign one or more of the values defined above to one or more of such dimensions (e.g., Action, Temporality, Certainty, and/or Actor). The dimensions represented by the Action, Certainty, Temporality, and/or Actor columns of table 502 can be orthogonal to each other (and therefore can comprise orthogonal dimensions), meaning the value of each aspect is independent of any other aspects.

FIGS. 6A, 6B, and 6C illustrate diagrams of example, non-limiting displays 600a, 600b, 600c that can facilitate summarization of medication events based on multidimensional medication event data extracted from a data source in accordance with one or more embodiments described herein. Repetitive description of like elements and/or processes employed in respective embodiments is omitted for sake of brevity.

Display 600a illustrated in FIG. 6A can comprise an example interactive visual display of one or more medication events and/or filter component 204 that can be generated by medication events summarization system 102 (e.g., via summarization component 202) in accordance with one or more embodiments of the subject disclosure described herein. Display 600a can comprise one or more panels. For instance, display 600a can comprise a top panel that can comprise a summary 602a of one or more medication events plotted in a timeline view. In another example, display 600a can comprise a bottom panel that can comprise an interactive filter 604a that can serve as an interactive visual display of filter component 204 to control the information flow into the visualization on the top panel, summary 602a.

The top panel, summary 602a, can present medication event information from structured and/or unstructured data in a timeline view. In FIG. 6A, the first horizontal bar 606a can represent the disease of interest (e.g., “Hypertensive Disorder, Systemic Arterial”) where one or more dots 608a on the bar can represent a clinical note. In some embodiments, this disease bar is not an element of display 600a. The disease bar allows medication events summarization system 102 to better describe (e.g., summarize) a sample use case. The other horizontal bars can each represent a medication (e.g., “Metolazone,” “Spironolactone,” “Carvedilol,” “Hydralazine,” “Furosemide,” “Amlodipine,” “Lisinopril,” “Clonidine,” etc.), where one or more dots 608a can represent a medication order from structured data, and dark gray bars 610a can represent when the medication was active or under administration as per structured medication orders. Each medication bar can also be activated (e.g., clicked) by an entity (e.g., an entity as defined above) to display a pop-up (e.g., text bubbles 612a) that can display how daily dosage has changed for the medication over time.

Display 600a can comprise text overlaid on the medication bars comprising text bubbles 612a that can represent medication events identified and classified from clinical notes (e.g., via extraction component 108). Each text bubble 612a can comprise: a description to show the value of each dimension for easy identification (e.g. “START|PAST|CERTAIN|PHYSICIAN”); text from the clinical note describing the event; and/or can be placed on the X-axis according to the date of the clinical note from which the event was extracted.

Display 600a can comprise dotted vertical lines 614a that can represent certain dates of interest corresponding to medication events. In practice, this would be a line that follows an entity's cursor to visually help the entity (e.g., an entity as defined above) line up various pieces of information in the display of display 600a. To reduce information overload, medication events summarization system 102 can allow such an entity to specify which medication(s) they would like to see events from clinical notes. As depicted in FIG. 6A, the deselected medications are greyed out with a dark gray overlay and no medication events from notes are displayed for the deselected medications. Display 600a illustrated in FIG. 6A depicts only medications related to the disease of interest (e.g., “Hydralazine”), but an entity implementing medication events summarization system 102 could hand-pick which combination of medications they want to see in the display.

The bottom panel, interactive filter 604a, can present several options across various dimensions to control what medication events from clinical notes an entity (e.g., an entity as defined above) would like to see displayed in the top panel, summary 602a. For example, the bottom panel, interactive filter 604a, can present a Source option, an Action option, a Temporality option, a Certainty option, and/or an Actor option, as illustrated in FIG. 6A.

SOURCE: This option allows an entity to control data flow from structured data (denoted as “Medication Order” in FIG. 6A) or unstructured data (denoted as “Clinical Notes” in FIG. 6A). If such an entity only selects Medication Order, all other dimensions are not available as they are only applicable for the data from clinical notes.

ACTION: This allows an entity to filter (e.g., via filter component 204) medication events from Clinical Notes across the action dimension. Such an entity can choose start, stop, increase, decrease and/or continue, or choose to see all the events. In some embodiments, each dimension can be represented by symbols.

TEMPORALITY: This allows an entity to filter (e.g., via filter component 204) medication events from Clinical Notes across the temporal dimension. Such an entity can choose past, current and/or future events, or choose to see all events.

CERTAINTY: This allows an entity to filter (e.g., via filter component 204) medication events from Clinical Notes across the certainty dimension. Such an entity can choose hypothetical, conditional and/or prescribed events, or choose to see all events.

ACTOR: This allows an entity to filter (e.g., via filter component 204) medication events from Clinical Notes across the actor dimension. Such an entity can choose patient, certain specialists (e.g., a primary care physician (PCP), a urologist, etc.), all physicians, or choose to see all events.

It should be appreciated that, depending on the use case and dataset, there is potential for additional values for each dimension that could be added to medication events summarization system 102. For example, ACTION can be expanded to also include non-dosage adjustment actions such as, for instance, changes from brand name to generic medications, or changes in timing of when to take medication (e.g., take in the morning instead of in the evening).

It should be appreciated that medication events summarization system 102 (e.g., via summarization component 202 and/or filter component 204) enables an entity (e.g., an entity as defined above), to summarize and/or customize: 1) which source of data to display (e.g., structured medication orders and/or unstructured clinical notes); 2) which medications to display events from structured data; 3) which medications to display events from unstructured data; and/or 4) what kinds of events from unstructured data to show based on Action, Temporality, Certainty, and Actor.

In an embodiment, display 600a illustrates an example use case of medication events summarization system 102 where an entity (e.g., an entity as defined above) wants to see all Current and Certain medication events from structured medication orders and unstructured clinical notes for the medication Hydralazine. In this example, to generate display 600a, such an entity can select (e.g., via an API, a REST API, a GUI, etc.) the following values.

Source: Medication Orders, Clinical Notes.

Action: All.

Temporality: Current.

Certainty: Certain.

Actor: Patient, All Physician.

Display 600a can be of interest to an entity trying to reconcile events from unstructured clinical notes with structured medication orders for a specific medication (e.g., “Hydralazine”) to develop a complete picture of what is actually happening in the patient.

Display 600b illustrated in FIG. 6B can comprise an example, non-limiting alternative embodiment of display 600a described above and illustrated in FIG. 6A. Display 600b can comprise a summary 602b and/or an interactive filter 604b that can respectively comprise example, non-limiting alternative embodiments of summary 602a and interactive filter 604a described above and illustrated in FIG. 6A. Summary 602b can comprise a first horizontal bar 606b, dots 608b, dark gray bars 610b, text bubbles 612b, and/or dotted vertical lines 614b that can respectively comprise example, non-limiting alternative embodiments of first horizontal bar 606a, dots 608a, dark gray bars 610a, text bubbles 612a, and dotted vertical lines 614a described above and illustrated in FIG. 6A.

In an embodiment, display 600b illustrates an example use case of medication events summarization system 102 where an entity (e.g., an entity as defined above) wants to see all Current and Certain medication events initiated by the Physician from structured medication orders and unstructured clinical notes for the medications Hydralazine and Furosemide (the text for these medications is not legible in FIG. 6B as they are positioned behind text bubble 612b). In this example, to generate display 600b, such an entity can select (e.g., via an API, a REST API, a GUI, etc.) the following values.

Source: Medication Orders, Clinical Notes.

Action: All.

Temporality: Current.

Certainty: Certain.

Actor: All Physician.

Display 600c illustrated in FIG. 6C can comprise an example, non-limiting alternative embodiment of display 600b described above and illustrated in FIG. 6B. Display 600c can comprise a summary 602c and/or an interactive filter 604c that can respectively comprise example, non-limiting alternative embodiments of summary 602b and interactive filter 604b described above and illustrated in FIG. 6B. Summary 602c can comprise a first horizontal bar 606c, dots 608c, dark gray bars 610c, text bubbles 612c, and/or dotted vertical lines 614c that can respectively comprise example, non-limiting alternative embodiments of first horizontal bar 606b, dots 608b, dark gray bars 610b, text bubbles 612b, and dotted vertical lines 614b described above and illustrated in FIG. 6B.

In an embodiment, display 600c illustrates an example use case of medication events summarization system 102 where an entity (e.g., an entity as defined above) wants to see all Current and Certain medication events initiated by the Patient from structured medication orders and unstructured clinical notes. In this example, to generate display 600c, such an entity can select (e.g., via an API, a REST API, a GUI, etc.) the following values.

Source: Medication Orders, Clinical Notes.

Action: All.

Temporality: Current.

Certainty: Certain.

Actor: Patient.

Based on the above example embodiments of displays 600a, 600b, 600c, it should be appreciated that an entity implementing medication events summarization system 102 can utilize filter component 204 to generate (e.g., via summarization component 202) one or more summaries (e.g., summaries 602a, 602b, 602c, etc.) based on one or more orthogonal dimensions selected by the entity using filter component 204. By utilizing filter component 204 to generate such one or more summaries based on at least one orthogonal dimension, such an entity can conveniently and quickly drill down to various medication events according to the desired use-case.

Medication events summarization system 102 can be associated with various technologies. For example, medication events summarization system 102 can be associated with medication reconciliation technologies, medical and/or healthcare records technologies, electronic health records technologies, electronic medical records technologies, machine learning technologies, artificial intelligence technologies, cloud computing technologies, and/or other technologies.

Medication events summarization system 102 can provide technical improvements to systems, devices, components, operational steps, and/or processing steps associated with the various technologies identified above. For example, medication events summarization system 102 can extract multidimensional medication events data corresponding to medication events from unstructured clinical notes and/or classify the medication events across multiple orthogonal dimensions. In another example, medication events summarization system 102 can further generate an interactive summary of the medication events that can be filtered by an entity based on at least one orthogonal dimension to conveniently and quickly generate at a point of care a comprehensive summary of medication events relevant to a certain use-case (e.g., a certain patient care management process).

Medication events summarization system 102 can provide technical improvements to a processing unit (e.g., processor 106, a quantum processor, etc.) associated with medication events summarization system 102. For example, by enabling an entity (e.g., an entity as defined above) to filter a summary of medication events generated by medication events summarization system 102 based on one or more orthogonal dimensions as described above, medication events summarization system 102 can thereby facilitate improved performance of such a processing unit (e.g., processor 106) by reducing the processing workload of the processing unit to generate a filtered interactive summary. In this example, by reducing the processing workload of such a processing unit (e.g., processor 106), medication events summarization system 102 can further facilitate improved processing efficiency and/or reduced computational costs of the processing unit (e.g., processor 106).

A practical application of medication events summarization system 102 is that it can be implemented by a healthcare provider (e.g., a paramedic, a physician) at the point of care (e.g., in an ambulance, in a hospital, etc.) to quickly generate a comprehensive chronological interactive summary of medication events of a patient based on multidimensional medication event data extracted from both structured data sources and unstructured data sources. Another practical application of medication events summarization system 102 is that it can be implemented by a healthcare provider (e.g., a paramedic, a physician) at the point of care (e.g., in an ambulance, in a hospital, etc.) to quickly generate a filtered and comprehensive chronological interactive summary of the medication events of a patient that can be tailored to a desired use-case by filtering the summary based on at least one orthogonal dimension to yield only information relevant to the desired use-case.

It should be appreciated that medication events summarization system 102 provides a new approach driven by relatively new electronic health records technologies. For example, medication events summarization system 102 provides a new approach to summarize medication-related events from a patient's electronic health record in a chronological order by extracting multidimensional medication event data from clinical notes and classifying the medication events across multiple orthogonal dimensions to capture various aspects of the medication events (e.g., action, temporality, certainty, actor, etc.). Using such dimensional information, medication events summarization system 102 can plot a chronological summary on a timeline with options (e.g., according to the various values in dimensions) for the user to adjust the information flow into the system (e.g., via filter component 204).

Medication events summarization system 102 can employ hardware or software to solve problems that are highly technical in nature, that are not abstract and that cannot be performed as a set of mental acts by a human. In some embodiments, one or more of the processes described herein can be performed by one or more specialized computers (e.g., a specialized processing unit, a specialized classical computer, a specialized quantum computer, etc.) to execute defined tasks related to the various technologies identified above. Medication events summarization system 102 and/or components thereof, can be employed to solve new problems that arise through advancements in technologies mentioned above, employment of quantum computing systems, cloud computing systems, computer architecture, and/or another technology.

It is to be appreciated that medication events summarization system 102 can utilize various combinations of electrical components, mechanical components, and circuitry that cannot be replicated in the mind of a human or performed by a human, as the various operations that can be executed by medication events summarization system 102 and/or components thereof as described herein are operations that are greater than the capability of a human mind. For instance, the amount of data processed, the speed of processing such data, or the types of data processed by medication events summarization system 102 over a certain period of time can be greater, faster, or different than the amount, speed, or data type that can be processed by a human mind over the same period of time.

According to several embodiments, medication events summarization system 102 can also be fully operational towards performing one or more other functions (e.g., fully powered on, fully executed, etc.) while also performing the various operations described herein. It should be appreciated that such simultaneous multi-operational execution is beyond the capability of a human mind. It should also be appreciated that medication events summarization system 102 can include information that is impossible to obtain manually by an entity, such as a human user. For example, the type, amount, and/or variety of information included in medication events summarization system 102, extraction component 108, classification component 110, summarization component 202, and/or filter component 204 can be more complex than information obtained manually by a human user.

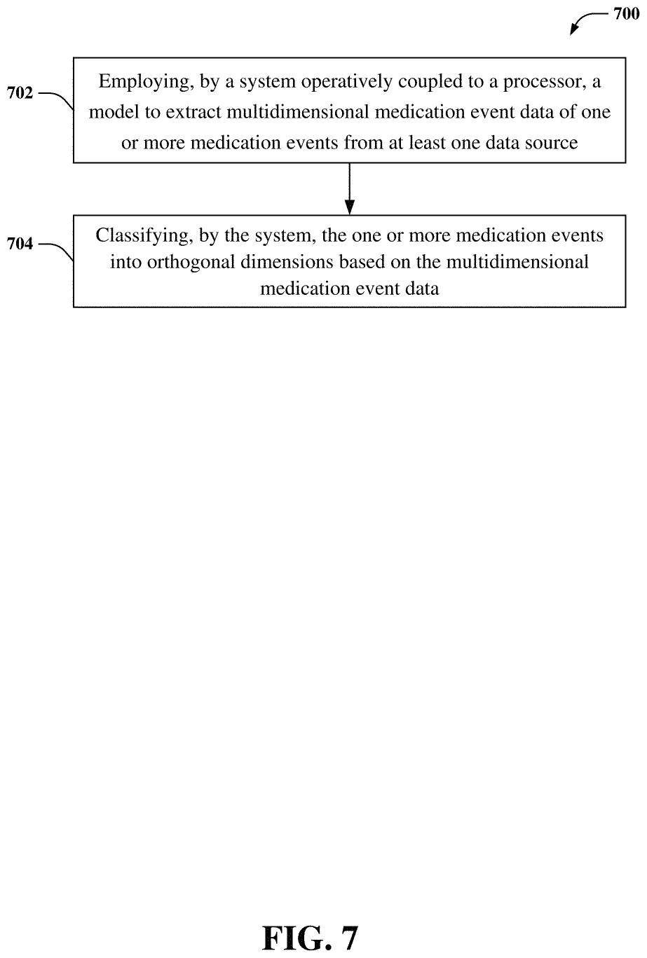

FIG. 7 illustrates a flow diagram of an example, non-limiting computer-implemented method 700 that can facilitate summarization of medication events based on multidimensional medication event data extracted from a data source in accordance with one or more embodiments described herein. Repetitive description of like elements and/or processes employed in respective embodiments is omitted for sake of brevity.

At 702, computer-implemented method 700 can comprise employing, by a system (e.g., via medication events summarization system 102 and/or extraction component 108) operatively coupled to a processor (e.g., processor 106, a quantum processor, etc.), a model (e.g., a BiLSTM-CRF model, a pretrained language model (e.g., transformer based) fine-tuning, etc.) to extract multidimensional medication event data (e.g., medication name, medication type, medication artifacts, medication sig, etc.) of one or more medication events (e.g., documented past, present, or future medication change) from at least one data source (e.g., structured medical orders, unstructured clinical notes, pharmacy filling data, etc.).

At 704, computer-implemented method 700 can comprise classifying, by the system (e.g., via medication events summarization system 102 and/or classification component 110) the one or more medication events into orthogonal dimensions (e.g., action, certainty, temporality, actor, etc.) based on the multidimensional medication event data.

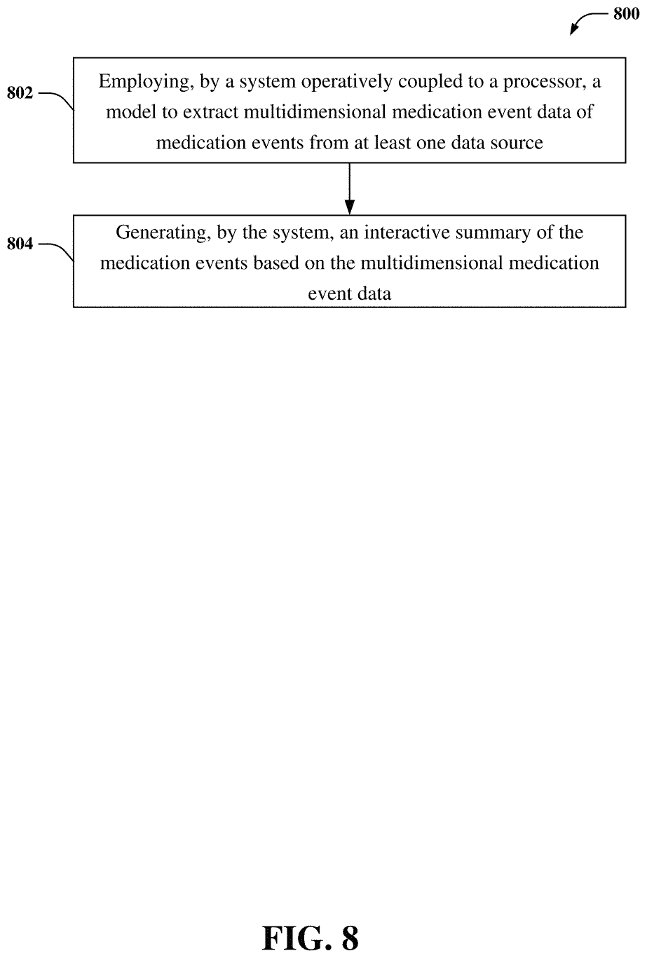

FIG. 8 illustrates a flow diagram of an example, non-limiting computer-implemented method 800 that can facilitate summarization of medication events based on multidimensional medication event data extracted from a data source in accordance with one or more embodiments described herein. Repetitive description of like elements and/or processes employed in respective embodiments is omitted for sake of brevity.

At 802, computer-implemented method 800 can comprise employing, by a system (e.g., via medication events summarization system 102 and/or extraction component 108) operatively coupled to a processor (e.g., processor 106, a quantum processor, etc.), a model (e.g., a BiLSTM-CRF model, a pretrained language model (e.g., transformer based) fine-tuning, etc.) to extract multidimensional medication event data (e.g., medication name, medication type, medication artifacts, medication sig, etc.) of one or more medication events (e.g., documented past, present, or future medication change) from at least one data source (e.g., structured medical orders, unstructured clinical notes, pharmacy filling data, etc.).

At 804, computer-implemented method 800 can comprise generating, by the system (e.g., via medication events summarization system 102 and/or summarization component 202) an interactive summary (e.g., summary 602a, 602b, 602c, etc.) of the medication events based on the multidimensional medication event data.

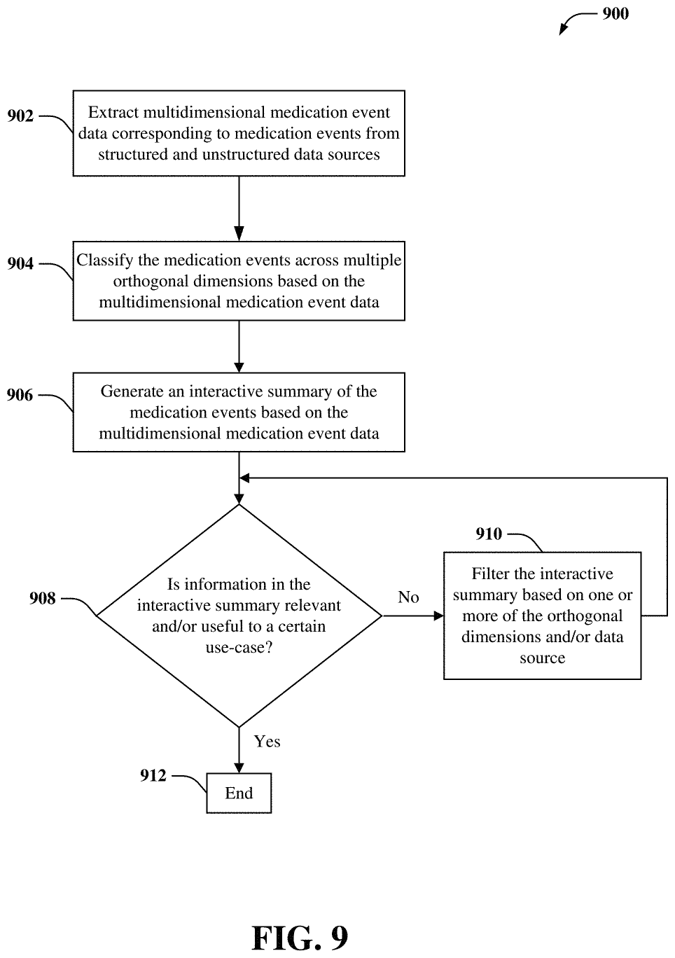

FIG. 9 illustrates a flow diagram of an example, non-limiting computer-implemented method 900 that can facilitate summarization of medication events based on multidimensional medication event data extracted from a data source in accordance with one or more embodiments described herein. Repetitive description of like elements and/or processes employed in respective embodiments is omitted for sake of brevity.

At 902, computer-implemented method 900 can comprise extracting (e.g., via medication events summarization system 102 and/or extraction component 108) multidimensional medication event data (e.g., medication name, medication type, medication artifacts, medication sig, etc.) corresponding to medication events (e.g., documented past, present, or future medication change) from structured and unstructured data sources (e.g., structured medical orders, unstructured clinical notes, pharmacy filling data, etc.).

At 904, computer-implemented method 900 can comprise classifying (e.g., via medication events summarization system 102 and/or classification component 110) the medication events across multiple orthogonal dimensions (e.g., action, certainty, temporality, actor, etc.) based on the multidimensional medication event data. For example, as described above with reference to FIG. 1, classification component 110 can employ a model (e.g., a classification model) to classify the medication events across such multiple orthogonal dimensions by classifying the multidimensional medication event data into such orthogonal dimensions.

At 906, computer-implemented method 900 can comprise generating (e.g., via summarization component 202) an interactive summary of medication events based on the multidimensional medication event data.

At 908, computer-implemented method 900 can comprise determining whether the information in the interactive summary is relevant and/or useful to a certain use-case (e.g., a certain patient care management process).

If it is determined at 908 that the information in the interactive summary is not relevant and/or useful to a certain use-case, at 910, computer-implemented method 900 can comprise filtering (e.g., via filter component 204) the interactive summary based on one or more of the orthogonal dimensions (e.g., action, certainty, temporality, actor, etc.) and/or data source (e.g., structured medication order, unstructured clinical notes, etc.).

If it is determined at 908 that the information in the interactive summary is relevant and/or useful to a certain use-case, at 912, computer-implemented method 900 can comprise ending.

For simplicity of explanation, the computer-implemented methodologies are depicted and described as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts can be performed to implement the computer-implemented methodologies in accordance with the disclosed subject matter. In addition, those skilled in the art will understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

In order to provide a context for the various aspects of the disclosed subject matter, FIG. 10 as well as the following discussion are intended to provide a general description of a suitable environment in which the various aspects of the disclosed subject matter can be implemented. FIG. 10 illustrates a block diagram of an example, non-limiting operating environment in which one or more embodiments described herein can be facilitated. Repetitive description of like elements employed in other embodiments described herein is omitted for sake of brevity.

With reference to FIG. 10, a suitable operating environment 1000 for implementing various aspects of this disclosure can also include a computer 1012. The computer 1012 can also include a processing unit 1014, a system memory 1016, and a system bus 1018. The system bus 1018 couples system components including, but not limited to, the system memory 1016 to the processing unit 1014. The processing unit 1014 can be any of various available processors. Dual microprocessors and other multiprocessor architectures also can be employed as the processing unit 1014. The system bus 1018 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI).

The system memory 1016 can also include volatile memory 1020 and nonvolatile memory 1022. The basic input/output system (BIOS), containing the basic routines to transfer information between elements within the computer 1012, such as during start-up, is stored in nonvolatile memory 1022. Computer 1012 can also include removable/non-removable, volatile/non-volatile computer storage media. FIG. 10 illustrates, for example, a disk storage 1024. Disk storage 1024 can also include, but is not limited to, devices like a magnetic disk drive, floppy disk drive, tape drive, Jaz drive, Zip drive, LS-100 drive, flash memory card, or memory stick. The disk storage 1024 also can include storage media separately or in combination with other storage media. To facilitate connection of the disk storage 1024 to the system bus 1018, a removable or non-removable interface is typically used, such as interface 1026. FIG. 10 also depicts software that acts as an intermediary between users and the basic computer resources described in the suitable operating environment 1000. Such software can also include, for example, an operating system 1028. Operating system 1028, which can be stored on disk storage 1024, acts to control and allocate resources of the computer 1012.

System applications 1030 take advantage of the management of resources by operating system 1028 through program modules 1032 and program data 1034, e.g., stored either in system memory 1016 or on disk storage 1024. It is to be appreciated that this disclosure can be implemented with various operating systems or combinations of operating systems. A user enters commands or information into the computer 1012 through input device(s) 1036. Input devices 1036 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1014 through the system bus 1018 via interface port(s) 1038. Interface port(s) 1038 include, for example, a serial port, a parallel port, a game port, and a universal serial bus (USB). Output device(s) 1040 use some of the same type of ports as input device(s) 1036. Thus, for example, a USB port can be used to provide input to computer 1012, and to output information from computer 1012 to an output device 1040. Output adapter 1042 is provided to illustrate that there are some output devices 1040 like monitors, speakers, and printers, among other output devices 1040, which require special adapters. The output adapters 1042 include, by way of illustration and not limitation, video and sound cards that provide a means of connection between the output device 1040 and the system bus 1018. It should be noted that other devices and/or systems of devices provide both input and output capabilities such as remote computer(s) 1044.

Computer 1012 can operate in a networked environment using logical connections to one or more remote computers, such as remote computer(s) 1044. The remote computer(s) 1044 can be a computer, a server, a router, a network PC, a workstation, a microprocessor based appliance, a peer device or other common network node and the like, and typically can also include many or all of the elements described relative to computer 1012. For purposes of brevity, only a memory storage device 1046 is illustrated with remote computer(s) 1044. Remote computer(s) 1044 is logically connected to computer 1012 through a network interface 1048 and then physically connected via communication connection 1050. Network interface 1048 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1050 refers to the hardware/software employed to connect the network interface 1048 to the system bus 1018. While communication connection 1050 is shown for illustrative clarity inside computer 1012, it can also be external to computer 1012. The hardware/software for connection to the network interface 1048 can also include, for exemplary purposes only, internal and external technologies such as, modems including regular telephone grade modems, cable modems and DSL modems, ISDN adapters, and Ethernet cards.

Referring now to FIG. 11, an illustrative cloud computing environment 1150 is depicted. As shown, cloud computing environment 1150 includes one or more cloud computing nodes 1110 with which local computing devices used by cloud consumers, such as, for example, personal digital assistant (PDA) or cellular telephone 1154A, desktop computer 1154B, laptop computer 1154C, and/or automobile computer system 1154N may communicate. Nodes 1110 may communicate with one another. They may be grouped (not shown) physically or virtually, in one or more networks, such as Private, Community, Public, or Hybrid clouds as described hereinabove, or a combination thereof. This allows cloud computing environment 1150 to offer infrastructure, platforms and/or software as services for which a cloud consumer does not need to maintain resources on a local computing device. It is understood that the types of computing devices 1154A-N shown in FIG. 11 are intended to be illustrative only and that computing nodes 1110 and cloud computing environment 1150 can communicate with any type of computerized device over any type of network and/or network addressable connection (e.g., using a web browser).

Referring now to FIG. 12, a set of functional abstraction layers provided by cloud computing environment 1150 (FIG. 11) is shown. It should be understood in advance that the components, layers, and functions shown in FIG. 12 are intended to be illustrative only and embodiments of the invention are not limited thereto. As depicted, the following layers and corresponding functions are provided:

Hardware and software layer 1260 includes hardware and software components. Examples of hardware components include: mainframes 1261; RISC (Reduced Instruction Set Computer) architecture based servers 1262; servers 1263; blade servers 1264; storage devices 1265; and networks and networking components 1266. In some embodiments, software components include network application server software 1267 and database software 1268.

Virtualization layer 1270 provides an abstraction layer from which the following examples of virtual entities may be provided: virtual servers 1271; virtual storage 1272; virtual networks 1273, including virtual private networks; virtual applications and operating systems 1274; and virtual clients 1275.

In one example, management layer 1280 may provide the functions described below. Resource provisioning 1281 provides dynamic procurement of computing resources and other resources that are utilized to perform tasks within the cloud computing environment. Metering and Pricing 1282 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 1283 provides access to the cloud computing environment for consumers and system administrators. Service level management 1284 provides cloud computing resource allocation and management such that required service levels are met. Service Level Agreement (SLA) planning and fulfillment 1285 provide pre-arrangement for, and procurement of, cloud computing resources for which a future requirement is anticipated in accordance with an SLA.

Workloads layer 1290 provides examples of functionality for which the cloud computing environment may be utilized. Non-limiting examples of workloads and functions which may be provided from this layer include: mapping and navigation 1291; software development and lifecycle management 1292; virtual classroom education delivery 1293; data analytics processing 1294; transaction processing 1295; and medication events summarization software 1296.

The present invention may be a system, a method, an apparatus and/or a computer program product at any possible technical detail level of integration. The computer program product can include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like.

The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices. For example, in one or more embodiments, computer executable components can be executed from memory that can include or be comprised of one or more distributed memory units. As used herein, the term “memory” and “memory unit” are interchangeable. Further, one or more embodiments described herein can execute code of the computer executable components in a distributed manner, e.g., multiple processors combining or working cooperatively to execute code from one or more distributed memory units. As used herein, the term “memory” can encompass a single memory or memory unit at one location or multiple memories or memory units at one or more locations.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components; and
a processor that executes the computer executable components stored in the memory, wherein the computer executable components comprise:
an extraction component that employs a model to extract multidimensional medication event data of one or more medication events from at least one data source; and
a classification component that classifies the one or more medication events into orthogonal dimensions based on the multidimensional medication event data.

2. The system of claim 1, wherein the at least one data source is selected from a group consisting of an electronic health record, an electronic medical record, a structured data source, a structured medication order, an unstructured data source, an unstructured clinical note, and pharmacy prescription filling data.

3. The system of claim 1, wherein the orthogonal dimensions are selected from a group consisting of action, temporality, certainty, and actor, and wherein the classification component classifies the one or more medication events into the orthogonal dimensions to improve accuracy of a summary of the one or more medication events generated by at least one of the system, the processor, or a medication reconciliation system.

4. The system of claim 1, wherein the computer executable components further comprise:
a summarization component that generates a summary of the one or more medication events based on the multidimensional medication event data.

5. The system of claim 1, wherein the computer executable components further comprise:
a filter component that filters at least one of a summary of the one or more medication events, the multidimensional medication event data, the one or more medication events, or the at least one data source based on at least one orthogonal dimension of the multidimensional medication event data.

6. A computer-implemented method, comprising:
employing, by a system operatively coupled to a processor, a model to extract multidimensional medication event data of one or more medication events from at least one data source; and
classifying, by the system, the one or more medication events into orthogonal dimensions based on the multidimensional medication event data.

7. The computer-implemented method of claim 6, wherein the at least one data source is selected from a group consisting of an electronic health record, an electronic medical record, a structured data source, a structured medication order, an unstructured data source, an unstructured clinical note, and pharmacy prescription filling data.

8. The computer-implemented method of claim 6, wherein the orthogonal dimensions are selected from a group consisting of action, temporality, certainty, and actor.

9. The computer-implemented method of claim 6, further comprising:
generating, by the system, a summary of the one or more medication events based on the multidimensional medication event data.

10. The computer-implemented method of claim 6, further comprising:
filtering, by the system, at least one of a summary of the one or more medication events, the multidimensional medication event data, the one or more medication events, or the at least one data source based on at least one orthogonal dimension of the multidimensional medication event data.

11. A computer program product facilitating a process to summarize medication events based on multidimensional medication event data extracted from a data source, the computer program product comprising a computer readable storage medium having program instructions embodied therewith, the program instructions executable by a processor to cause the processor to:
employ, by the processor, a model to extract multidimensional medication event data of one or more medication events from at least one data source; and
classify, by the processor, the one or more medication events into orthogonal dimensions based on the multidimensional medication event data.

12. The computer program product of claim 11, wherein the at least one data source is selected from a group consisting of an electronic health record, an electronic medical record, a structured data source, a structured medication order, an unstructured data source, an unstructured clinical note, and pharmacy prescription filling data.

13. The computer program product of claim 11, wherein the orthogonal dimensions are selected from a group consisting of action, temporality, certainty, and actor.

14. The computer program product of claim 11, wherein the program instructions are further executable by the processor to cause the processor to:
generate, by the processor, a summary of the one or more medication events based on the multidimensional medication event data.

15. The computer program product of claim 11, wherein the program instructions are further executable by the processor to cause the processor to:
filter, by the processor, at least one of a summary of the one or more medication events, the multidimensional medication event data, the one or more medication events, or the at least one data source based on at least one orthogonal dimension of the multidimensional medication event data.

16. A system, comprising:
a memory that stores computer executable components; and
a processor that executes the computer executable components stored in the memory, wherein the computer executable components comprise:
an extraction component that employs a model to extract multidimensional medication event data of medication events from at least one data source; and
a summarization component that generates an interactive summary of the medication events based on the multidimensional medication event data.

17. The system of claim 16, wherein the at least one data source is selected from a group consisting of an electronic health record, an electronic medical record, a structured data source, a structured medication order, an unstructured data source, an unstructured clinical note, and pharmacy prescription filling data.

18. The system of claim 16, wherein the summarization component further determines daily medication dosage data based on the multidimensional medication event data.

19. The system of claim 16, wherein the computer executable components further comprise:
a classification component that classifies the medication events into orthogonal dimensions based on the multidimensional medication event data to improve at least one of accuracy of the interactive summary or performance of at least one of the system, the processor, the summarization component, or a medication reconciliation system, and wherein the orthogonal dimensions are selected from a group consisting of action, temporality, certainty, and actor.

20. The system of claim 16, wherein the computer executable components further comprise:
a filter component that filters at least one of the interactive summary, the multidimensional medication event data, the medication events, or the at least one data source based on at least one orthogonal dimension of the multidimensional medication event data, thereby facilitating improved performance of the processor in generating the interactive summary.

21. A computer-implemented method, comprising:
employing, by a system operatively coupled to a processor, a model to extract multidimensional medication event data of medication events from at least one data source; and
generating, by the system, an interactive summary of the medication events based on the multidimensional medication event data.

22. The computer-implemented method of claim 21, wherein the at least one data source is selected from a group consisting of an electronic health record, an electronic medical record, a structured data source, a structured medication order, an unstructured data source, an unstructured clinical note, and pharmacy prescription filling data.

23. The computer-implemented method of claim 21, further comprising:
determining, by the system, daily medication dosage data based on the multidimensional medication event data.

24. The computer-implemented method of claim 21, further comprising:
classifying, by the system, the medication events into orthogonal dimensions based on the multidimensional medication event data, wherein the orthogonal dimensions are selected from a group consisting of action, temporality, certainty, and actor.

25. The computer-implemented method of claim 21, further comprising:
filtering, by the system, at least one of the interactive summary, the multidimensional medication event data, the medication events, or the at least one data source based on at least one orthogonal dimension of the multidimensional medication event data.
